(19) United States
(12) Patent Application Publication
(10) Pub. No.: US 2005/0171460 A1
(43) Pub. Date: Aug. 4, 2005

(54) Variable arm abduction orthosis

(76) Inventor: Karin Behr, Regensburg (DE)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(21) Appl. No.: 10/506,000

(22) PCT Filed: Feb. 25, 2003
(86) PCT No.: PCT/EP03/01926

(30) Foreign Application Priority Data
Feb. 27, 2002 (DE) 102 08 406.8

(51) Int. Cl. A61F 5/00

(52) U.S. Cl. 602/20

(57) ABSTRACT

The invention relates to a variable support of the arm add/or the shoulder The device of the invention can especially be used for the conservative or postoperative treatment of various injuries of the shoulder. Preferably, the inventive device comprises a distal element (1), a proximal element (2) and an axillary wedge (3) that can be adjusted relative to the distal element (1) and/or the proximal element (2) at least in height. In a further embodiment of the invention, the distal element can be adjusted relative to the proximal element at least in angle and in a third preferred embodiment, both the axillary wedge and the distal element can be adjusted. The invention is specifically advantageous in that the orthosis can be variably adjusted without complication in accordance with the size of the patient and the medical requirements.

The present invention relates to a device for variably supporting the arm and/or the shoulder. Devices of this kind are used for the conservative or postoperative treatment of different kinds of shoulder injuries. In case of an injury of the shoulder, it is desirable to protect the result of an operation, to avoid complications and reduce occurring pain by muscular relaxation. Moreover, an appropriate position of the shoulder joint has to be assured to create the basis for physiologic movements.

Depending on the extent of damage, which can often only be noticed during an operation, or depending on the extent of the operative procedure, the operating surgeon can often determine the adequate support of the arm in the operating room only. Depending on the above mentioned factors, the appropriate angle position of the arm is defined. i.e. the operating surgeon prescribes the optimum abduction angle of the arm.

There are different kinds of devices for the treatment of shoulder/arm injuries, which can be divided into the groups of immobilization bandages, arm abduction support wedges or cushions and arm abduction splints. These groups will be described in the following:

1. Immobilization Bandages

Examples of immobilization bandages are the TRICODOR Gilchrist's bandage of the company Beiersdorf AG and the OMOTRAIN of the company Bauerfeind. Such mostly elastic bandages closely contact the body and are intended to fix the shoulder and the arm by means of slings around the upper part of the body, the hip, the waist, the arm and/or the shoulder. They are used for the treatment of postoperative and posttraumatic indications on the shoulder and the upper arm.

The application of immobilization bandages turned out to be uncomfortable and non-functional, because the fixing of the arm in an extremely inwardly rotated position, which is caused by said bandage, can easily lead to a conglutination of the recessus axillaris and thus to an undesired contracture in the shoulder joint. Moreover, already within one or two days, the immobilization of the elbow joint at an angle of 90° can lead to a severe hypertonia of the m. biceps brachii and, concomitantly, to perception disorders in the area of the m. ulnaris. Patients regularly complain of having perception disorders in the fourth and fifth fingers.

Moreover, immobilization bandages hinder the patient in adequately putting on his/her clothes and cause hygiene problems.

2. Abduction Wedges and Cushions

Known embodiments are abduction cushions of the company Orthopädietechnik Kurtze GmbH, the DonJoy armsling ULTRA SLING of the company medi Bayreuth, the inflatable medi shoulder abduction cushion SAK of the company medi Bayreuth as well as the abduction support wedge LÜBECK of the company ADEV Orthopädiebedarf. By means of a sling, the abduction wedges or cushions are hung transversely across the shoulder of the patient and are supported by the waist or the iliac crest on the opposite side of the patient's body. The lower arm of the patient is fixed to the wedge or cushion by means of slings

In one out of a plurality of embodiments, DE-U-29717429 describes a bandage system comprising an abduction cushion which is divided in the longitudinal direction and consists of two molded parts. The molded parts are connected with each other via elastic ribbons extending over the top and bottom, wherein intermediate pieces, which are made of the same material as the abduction cushion and serve the purpose of changing the distance, can be introduced into the spaces formed between the two molded parts in order to change the abduction angle.

Although these abduction cushions are mostly more comfortable for the patient than the Gilchrist's bandage, they do not meet the requirements of the medical specialists because they hardly take into account the biomechanics, the function of the shoulder joint and the operative result.

3. Arm Abduction Splints

DE-A-1 280 471 describes an adjustable abduction splint comprising a support part which can be locked by means of tension members on the left and Tight sides of a wearer's body and to which a splint for the upper arm is attached in a lockable manner via a vertically and horizontally movable axillary joint. The split for the upper arm is attached in a horizontally rotatable and lockable manner attached to a wing of a hinge that can be swiveled in a vertical plane, and the other wing is connected to the support part. The angle between the wings of the binge can be locked by means of a strut whose length is adjustable and whose ends are attached to the support part and/or the wing of the hinge.

Further known embodiments of abduction splints are ABDUKTOR, System Dr.

Berrehail, of the company Polytech Silimed Europe GmbH, the modular arm abduction orthosis of the company Otto Bock, Orthopädische Industrie, QUADRANT of the company medi Bayreuth, S.C.O.I. of the company medi Bayreuth as well as ARTROSWING of the company Owned Medizintechnik. The arm abduction splints are adjustable splints which are attached to the upper part of the body by means of a plurality of straps and which fix the arm in a position. In some embodiments, the position of the splint is variable depending on the kind and state of the injury.

Although abduction splints more or less meet the requirements of medical specialists, they only insufficiently take into account the patient's feeling of well-being. When being used, abduction splints create much trouble for the patient, in particular during the night Moreover, the patient can only very rarely put on these splints himself/herself Experience shows that patients wear these very expensive abduction splints only rarely or not at all.

The demands on an optimum arm abduction orthosis can be divided in three groups. The first group comprises the doctor's demands, the second group the patient's demands, and the third group the physiotherapist's demands on an arm abduction orthosis.
(a) The doctor demands an arm abduction orthosis with variably adjustable arm length and with selectable abduction angle, which can be put on quickly and easily directly after the operation in the recovery room. On the one hand, this guarantees a reduction of costs because the provisional immobilization bandage (e.g. Gilchrist's bandage) is not necessary. On the other hand, much time can be saved because, in contrast to the conventional, technically complicated abduction splints, no sanitary engineer is necessary for putting on the splints, and the doctor himself/herself can easily fix the orthosis when the patient is still narcotized.
(b) The patient demands an arm abduction orthosis which is very comfortable to wear, offers safety during night and day and can be put on and off alone without lie aid of a third person. Moreover, the orthosis has to fix the shoulder joint in a position which avoids shrinking of the joint capsule and thus the painful restricted movement.
(c) The physiotherapist demands an arm abduction orthosis which can at any time be adjusted by the patient and the therapist to the present degree of movement, depending on the pain, the healing of the operated structures as well as the worked-out rehabilitation result. Moreover, the orthosis has to allow a functional treatment as early as possible, because the latter only requires a short treatment for restoring the joint mobilization. Furthermore, the shoulder joint has to be protected when the ellbow and wrist joints are moved, so that the postoperative edema can be reduced Even if an arm orthosis is put on, it has to be possible to use the hand for any ordinary movements, e.g. for eating.

In order to avoid the disadvantages of existing solutions and for better meeting the described demands on an arm abduction orthosis, the inventor first further developed an arm abduction cushion. This orthosis consists of a base cushion with an abduction angle of about 30°, which can be enlarged to a larger abduction angle by means of an enlargement element. The individual elements are connected with each other by means of book and loop fastening tapes.

The advantages of this solution reside in that especially the patients accept it more readily since it is more comfortable to wear and costs are saved because the abduction orthosis can be put on the patient directly in the operating room. In the latter case, the doctor can choose between two abduction angles. It is a disadvantage of this first development step that the adaptability to the patient's arm length is insufficient. There might be problems to put on and adapt the arm abduction orthosis to patients being shorter than 160 cm, i.e. patients having a short upper arm, or to very tall patients being taller than 180 cm. Moreover, it is not possible that the doctor, patient or physiotherapist can flexibly adjust the abduction angle in several abduction degrees depending on the operative treatment, the pain and the rehabilitation process. Moreover, it turned out that the plexus axillaris becomes irritated when patients are treated with the 60° arm abduction orthosis, because when using this treatment the wider axillary wedge has to be used for enlarging the abduction angle. Moreover, female patients having a large bust size complain about an insufficient wearing comfort.

It is an object of the invention to provide an improved device for supporting the arm and/or shoulder. This object is achieved with the subject-matter of the claims.

The present invention meets the above-mentioned requirements and overcomes the drawbacks of the state of the art.

The invention is based on the concept of providing a device for supporting the arm and/or shoulder, comprising an arm fixing element located distant from the body (distal element), as well as an axillary wedge and/or an element located adjacent to the body (proximal element), wherein the axillary wedge is adjustable at least in height by means of an elongation element, and/or the distal element and the proximal element can be adjusted as regards their distance and/or angle.

In a preferred embodiment, the elongation element for adjusting the axillary wedge is a mechanical element, preferably a telescopic aluminum rod which, more preferably, can be locked continuously or almost continuously with relatively little distance between the serrations. A telescopic rod can preferably be locked by means of a stepped catch (e.g., as in a headrest of a vehicle) or other catches, e-g. by means of a thumb nut. In a preferred embodiment, the axillary wedge is furthermore adjustable in angle and height relative to the distal element, replaceable and/or variable in its outer shape. Preferably, the axillary wedge is adjustable in angle relative to the distal element and/or the proximal element in a range of ±15°.

For achieving an as universal adjustability as possible, a ball-and-socket joint is preferably provided between the elongation element and the axillary wedge. A ball-and-socket joint is advantageous in that it can be locked completely or, in the almost locked state, the axillary wedge is allowed to slightly move or yield when a specific minimum force is applied. The wearer himself/herself can therefore adjust or adapt the device to his/her demands as desired, wherein the supporting function is maintained.

The axillary wedge preferably has a dorsally raised shape. As a rule, it is not necessary to increase the width of the axillary wedge because, on the one hand, the contact surface of the lower arm is sufficient for increasing the abduction angle and, on the other hand, a smaller wedge provides for an increased wearing comfort Nevertheless, in some cases an exchangeable wedge or a wedge whose outer shape can be altered might be advantageous for an optimum adaptation.

The invention further relates to a device for supporting the arm and/or the shoulder comprising a distal element and a proximal element attached thereto. The distal element and/or the proximal element is/are cushioned at least in outer portions and, moreover, essentially rigid at least in inner portions, e.g. by means of plastic or metal plates. An angle adjusting means for adjusting the angle between the distal element and the proximal element is arranged in the area of the inner portions. Thus, a comfortable, easy and nevertheless defined support can be achieved.

The distal and proximal elements are preferably connected by means of a hinge or joint connection, wherein in further preferred embodiment, the connection can be realized by means of a joint, a hinge, a strap hinge, a web of fabric, a fixed intermediate roll or also simply by sewing. An intermediate roll being attached, or fixed by means of a hook and loop tape, to the distal and proximal elements is particularly advantageous because, on the one hand, it is favorably priced and, on the other hand, also allows an effectively spaced-apart connection between these elements and has at the same time a cushioning effect.

The distal and proximal elements are preferably adjusted relative to each other mechanically or pneumatically. This can be done, e.g., by mechanically adjustable joints or telescopic rods and/or by pneumatically adjustable cylinders or inflatable elements. The distal and proximal elements are adjustable preferably in an angle of at least about 30° to 60°, and in the base state they are at an angle of 30° relative to each other. Here, the outer angle is meant, i.e. the angle between the outer surfaces of the distal element and the proximal element. More preferably, in accordance with the invention it is possible to adjust the angle up to about 90°. in a further preferred embodiment, the inner angle between the distal element and the proximal element, i.e. the angle between their inner surfaces, can be reduced to about 0°. The outer base angle of 30° between the effective outer surfaces of the distal element and the proximal element is preferably achieved by the wedge shape of the distal element.

A particularly preferred angle adjusting means is a spreading, means which, for spreading the distal and proximal elements, comprises a threaded spindle which can be rotated by a user, a nut being axially rotatable on the threaded spindle by rotating the threaded spindle, as well as arms attached to the nut and also to the distal and/or proximal elements. Self-locking threads, such as fine-pitch threads, are realized between the threaded spindle and the nut.

A grip by means of which a user can handle the free end of the threaded spindle is preferably provided at the end of the threaded spindle. This grip is preferably a star-shaped grip, so that the handling thereof is improved However, this grip can also have other features improving the handling thereof, e.g. a knurl.

To each of the upper ends of the distal element and the proximal element, one hinge element, which preferably has a support for stationarily and rotationarily supporting the threaded spindle, is attached More preferably, the hinge element is a double hinge joint, i.e. comprising a joint for the distal element and a joint for the proximal element.

Finally, the distal element and the proximal element preferably comprise cavities, so that in the folded state there is enough room for the end of the threaded spindle.

A further preferred embodiment is an engaging or latching means, similar to a means known, e.g., from the manufacture of furniture for engaging or latching loungers or beds in an upward position. The two main legs of the engaging or latching means are firmly attached to the inner portions of the distal element and the proximal element. The angle is adjusted in that a latch element engages in or clicks into teeth; it can then be increased in little steps in accordance with the tooth pitch and/or decreased after a slight increase and after disengagement, and optionally the means can be disengaged in a maximum position by increasing the angle and can then be folded again.

Alternatively, it is possible to provide an angle element which is adjustable between the distal element and the proximal element and which can be attached firmly and detachably, e.g. via hook and loop closures, to at least an inner portion of the distal element and/or proximal element, preferably to both.

Moreover, the angle adjusting means can comprise a link adjusting means which is, i.a., advantageous in that an angle scaling can easily be included.

The angle adjustment, height adjustment, patient's arm length and/or the abduction angle can preferably be read off and/or selected directly and/or indirectly via corresponding, scales. Moreover, the length and angle can preferably be adjusted continuously or almost continuously. For allowing an optimum, repeatable adjustment of the orthosis (abduction angle), the latter comprises scales and/or marks, preferably for measuring and/or adjusting the angle and length. Such scales can be applied to existing elements of the device, e.g. to telescopic rods or joints, or they can also be attached in a non-removable, removable or exchangeable manner to the orthosis as additional elements. In principle, all common measuring principles are suitable for the measurement, wherein the measurement can be carried out both directly and indirectly. In a preferred embodiment, the plurimeter measuring system is used for measuring the angle. In a further preferred embodiment, the desired adjustment of the orthosis can be made directly via the patient's parameters. In this case, geometrical conversions are already considered in the scale values. A continuous definition of the patient's parameters is important in this regard, e.g. the length of the upper arm as the distance between the lateral acromion tip and the head of the radius.

When adjusting the angle of the proximal element relative to the distal element, the adjustment preferably should not lead to a rising of the arm rest, e.g. by the formation of a step at the upper sides of the elements. This can be guaranteed, e.g., by providing an axis of rotation below the upper edge of the distal element and/or at the proximal element in an inwardly displaced manner.

In practice, care must first be taken that the patient's relieving posture (elevation of the shoulder) is compensated for during the rehabilitation. The relieving posture is caused, e.g., by pain or as a compensation, in order to compensate for the lacking muscular power in the shoulder by a hypertonia of the neck muscles. It is aimed at achieving a symmetric height of both shoulders. Moreover, the elbow should lie in a provided, extremely softly padded cavity of the cushion, so that the n. ulnaris is protected. In a further preferred embodiment, the individual elements are therefore flexible so that they adjust to the specific anatomy of the patient For this purpose, the elements are preferably padded and preferably filled with a foam material having a memory effect (relax foam) in particular in the area of the n. ulnaris, so that they adjust in shape to the patient and keep the undergone deformations over a long time. The foam material has preferably open cells so as to be breathable.

In order to improve the wearing comfort and the patient's hygiene, the elements of the device preferably have a breathable cover, particularly preferably a vapor permeable and liquid repelling cover material which can be attached permanently or by means of a hook and loop closure and particularly preferably to the inner portions of the distal and proximal elements.

The invention furthermore relates to a set including the above-mentioned device, wherein the latter comprises at least the first, above-mentioned cover and, moreover, at least a further cover, mainly for treating the patient directly after an operation, i.e. for post-operatively wearing the device, wherein the further cover is at least liquid repelling After a certain time has lapsed, and in particular if there is no longer the risk that the first cover becomes dirty by the operation or the patient's wound, the further cover can be removed, preferably by simple attachment means such as hook and loop closures. If the first cover is a permanent cover, the further cover is dimensioned such that it fits over the first one.

In a preferred embodiment of the invention, the two cushions have a slight C-shape which is adapted to the physiogonomy of the body. The concave side contacts the body. Moreover, in a preferred embodiment of the invention, the distal and proximal elements have a height of 16 cm and the axillary wedge has a height of 8 cm. Moreover, the width of the distal element is preferably 10 cm. The lower rear width of the proximal element is preferably 8 cm, and the lower front width is 5 cm, and the proximal element tapers towards the top to a width of about 0.5 to 1 cm and/or ends with a corresponding radius. In this embodiment, the axillary wedge has a width of about 10 cm. The preferred length of the orthosis is 40 cm. Depending on the concrete embodiment, the embodiments of the invention can have parameters differing from the above-mentioned ones.

In a further preferred embodiment, the wearing and closing mechanisms allow an easy handling Moreover, the device has preferably at least one flap or loop for fixing the arm, wherein preferably the edges of the flap or loop are padded in order to increase the wearing comfort. The device can preferably be combined with a hand training device.

In the following, preferred embodiments of the invention are described in a non-restricting and exemplary manner.

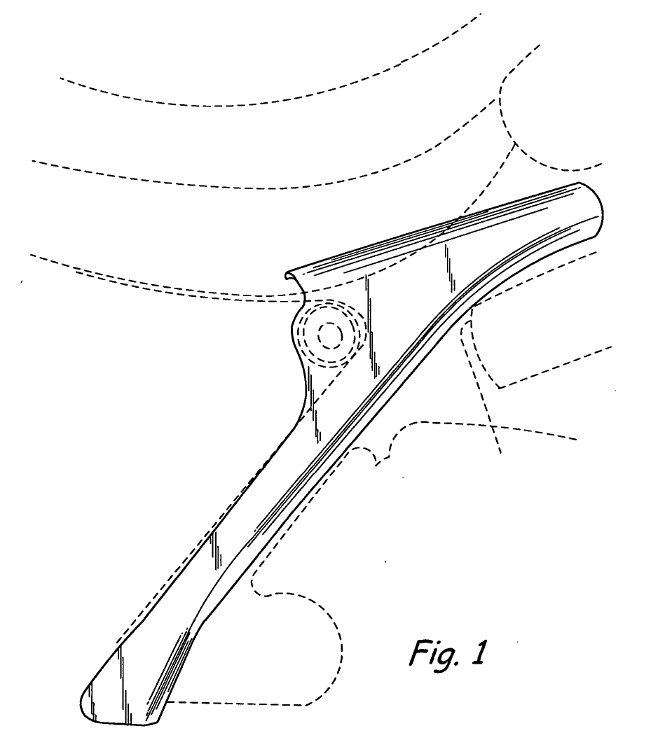

FIG. 1a is a spatial view of a preferred embodiment of a device of the invention from the back and from outside;

FIG. 1b is a representation similar to that of FIG. 1a with an elongated axillary support;

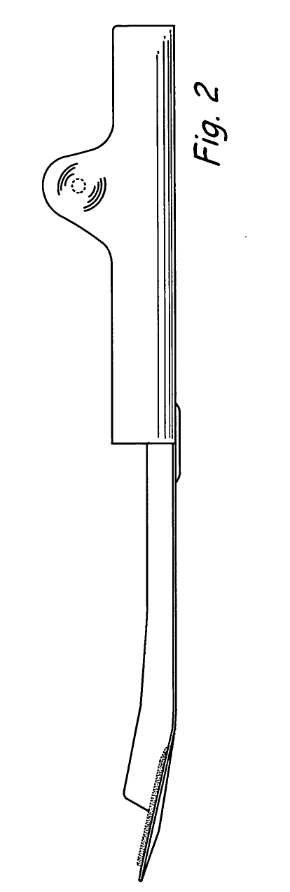

FIG. 2 shows a preferred embodiment of the invention similar to that of FIG. 1a with an angle-adjustable proximal element; and

FIG. 3a is a front view of a preferred embodiment of the invention on a patient,

FIG. 3b is a front view of a preferred embodiment of the invention with a height-adjustable axillary support,

FIG. 3c is a front view similar to that of FIG. 3b with an angle-adjustable proximal element,

FIG. 3d is a side view of a preferred embodiment of the invention,

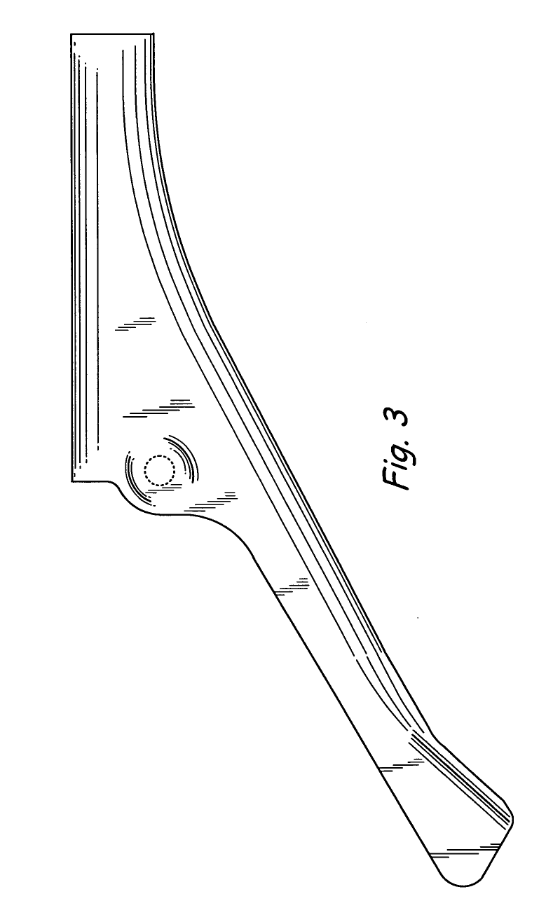

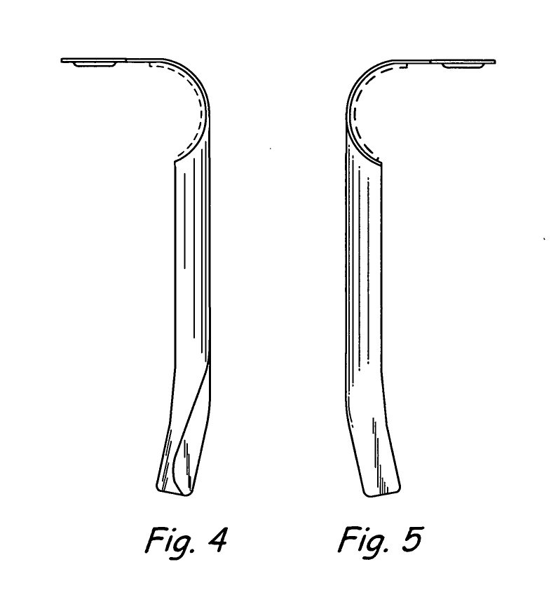

FIG. 4 is a spatial view of the device of the invention similar to that of FIG. 1 with an angle scale,

FIG. 5 is a spatial view of the device of the invention similar to that of FIG. 1 with a plurimeter measuring system,

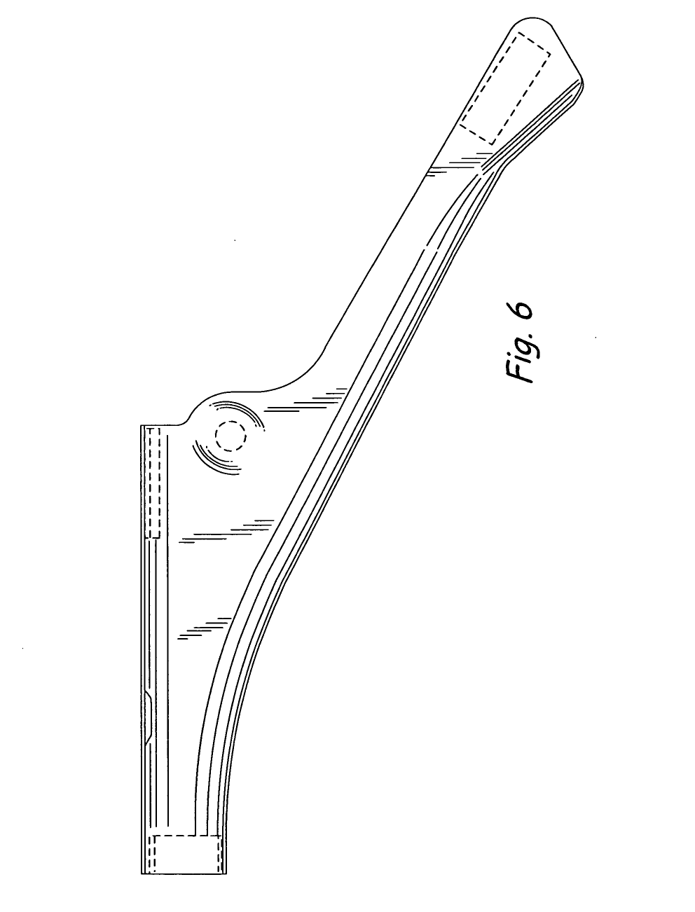

FIG. 6a is a front view of a preferred embodiment of the invention with an angle-adjustable axillary support,

FIG. 6b is a front view of a preferred embodiment of the invention with a system for measuring the axillary support adjustment,

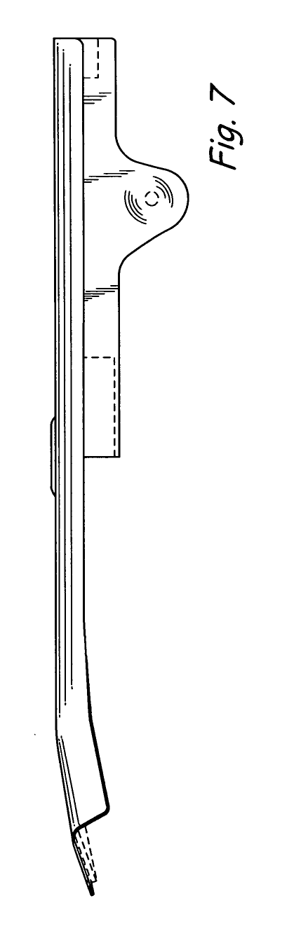

FIG. 7a is a side view of a further embodiment of the invention with an engaging or latching means,

FIG. 7b is a side view similar to that of FIG. 7a with more widely opened distal and proximal elements,

FIG. 7c is a side view of the engaging or latching means which is already shown in FIGS. 7a and 7b in combination with tile distal and proximal elements.

FIG. 7d is a side view of the engaging or latching means similar to that of FIG. 7c but in a more widely opened position,

FIG. 8a is a side view of a further embodiment of the invention with a movable angle adjusting means,

FIG. 8b is a side view similar to that of FIG. 8a with more widely opened distal and proximal elements,

FIG. 8c is a side view of a further embodiment of the invention with a lower link adjusting means,

FIG. 8d is a side view similar to that of FIG. 8c with more widely opened distal and proximal elements,

FIG. 8e is a bottom view of a detail of the link adjusting means of FIG. 8c,

FIG. 8f is a side view of a further embodiment of the invention with an angle adjusting means having a lever/latch mechanism,

FIG. 8g is a side view similar to that of FIG. 8f with more widely opened distal and proximal elements,

FIG. 8h is a side view of a further embodiment of the invention with a lateral link adjusting means,

FIG. 8i is a side view similar to that of FIG. 8h with more widely opened distal and proximal elements,

FIG. 9a is a side view of a further embodiment of the invention with a spreading means, and

FIG. 9b is a side view similar to that of FIG. 9a with more widely opened distal and proximal element.

In the following description of examples of preferred embodiments of the invention, elements having the same technical effect have the same reference numerals.

FIG. 1a shows a variable arm abduction cushion comprising a distal element 1, a proximal element 2 and an axillary wedge 3. By means of an elongation element 4, the axillary wedge 3 is connected with the proximal element 2 or preferably with the distal element 1 so as to be adjustable at least in height, preferably in height and angle. An arm fixing device 5 for holding the arm is provided on the distal element 1. The arm fixing device 5 preferably comprises a fixing element 6, which is preferably configured sa, a cloth, as well as of hook and loop tapes and holding straps or belts 7. In a preferred embodiment of the invention, the distal element 1 and the proximal element 2 are connected with each other by means of a hinge-like or joint-like element 8. Moreover, the variable arm abduction orthosis comprises devices 9, preferably buckles or eyelets, for attaching the arm fixing device 5 by means of the holding straps 7.

FIG. 1b shows an embodiment of the invention according to FIG. 1a, wherein the auxiliary wedge 3 is adjustable in height relative to the distal and proximal elements 1 and 2, respectively, by means of two elongation elements 4.

FIG. 2 shows an embodiment of the invention according to FIG. 1, wherein the proximal element 2 is inclined relative to the distal element 1 by an angle α.

FIG. 3 shows front and side views of an embodiment of the variable arm abduction orthosis of the invention. FIG. 3a shows the orthosis in a preferred position on the patient. The outer side of the proximal element 2 laterally contacts the upper part of the body of the patient 10. The axillary wedge 3 is positioned under the patient's axilla. The lower arm 11 of the patient contacts the outer side of the distal element. In a preferred embodiment of the variable arm abduction orthosis, the distal element has a cavity 12 for an improved support of the lower arm 11. FIG. 3b shows an embodiment of the invention comprising an axillary wedge 3 that is adjustable in height by means of the mechanical adjusting element 4, preferably a telescopic rod. FIG. 3c furthermore shows a proximal element 2 which is inclined relative to the distal element 1 by an angle α. The angle adjustment is performed, e.g., by means of an angle adjusting means 13, which is a mechanical or pneumatic means in accordance with preferred embodiments, as well as by means of a joint-like connection 8, which, according to preferred embodiments, is configured as a mechanical joint, straps, belts or connections made of fabric. FIG. 3d is a side view of a preferred embodiment of the invention with a hand training device 14.

FIG. 4 shows an embodiment of the invention according to FIG. 1, wherein, a measuring element 15 between the proximal element 2 and the distal element 1 shows the respective angle adjustment, the respective abduction angle and/or other parameters via a scale and/or wherein these parameters can be adjusted via this scale.

FIG. 5 shows an embodiment of the invention according to FIG. 1., wherein the angle adjustment and/or the respective abduction angle can be read from and/or selected via a plurimeter 16 attached to the proximal element 2.

FIG. 6 shows a front view of an embodiment of the variable arm abduction orthosis of the invention. FIG. 6a shows the orthosis with an axillary wedge 3 that can be adjusted in angle, e.g., via a ball-and-socket joint. FIG. 6b shows the orthosis with a scale 17 attached to the adjusting element 4 for the height adjustment and serving the purpose of adapting the parameters of the orthosis to the patient and/or adjusting the orthosis to the length of the patient's upper arm. In the latter case, the selected abduction angle is included in the scale 17.

FIG. 7a shows a side view of a further embodiment of the invention with an engaging or latching means 13a. At first, it is evident that the distal element 1 has an essentially rigid, stiff or strong inner portion la and the proximal element 2 has a corresponding inner portion 2a being equally or similarly stiff. The stiffness is particularly evident relative to the padded outer portions 1b, 2b. While padded outer portions 1b, 2b are to guarantee a comfortable support, the inner portions la, 2a are to guarantee a sufficiently stable support of the angle adjusting means 13. The inner portions 1a, 2a preferably extend only over a required length and width of the angle adjusting device. Thus, on the one hand, weight is saved and, on the other hand, a sufficiently stable device is provided.

Moreover, a cover 1c for the distal element 1 and a cover 2c for the proximal element, which were described above, are shown.

It is also evident from FIG. 7a that the left-hand end of the inner portion 2b is configured such that also the adjusting element 4 for the axillary wedge 3 is sufficiently stably supported.

FIG. 7a furthermore shows an intermediate roll 8 which joins the distal element 1 and the proximal element 2 in a hinge-like manner. Moreover, an axillary padding 3a, preferably made of so-called relax foam with memory characteristics, and a ball-and-socket joint 3b are shown between the adjusting element 4 and the axillary wedge 3. As already described above, the ball-and-socket joint 3b guarantees a fixing of the axillary wedge 3 that is flexible and optionally elastic under relatively great load.

FIG. 7b shows the front view of FIG. 7a in a more widely opened state. The functioning of the engaging or latching means 13a is particularly evident from FIGS. 7c and 7d. FIG. 7c shows the engaging or latching means 13a in the position closed to the greatest possible extent, i.e. preferably defining an angle position of 30° between the distal element 1 and the proximal element 2. In contrast, FIG. 7d shows a widely opened position with an angle of about 90° or close to 90°. In the assembled state, the first leg 13a¹ is attached to the distal element 1. The second leg 13a² is accordingly attached to the proximal element 2. A sleeve 13a⁴ having a mandrel 13a⁵, via which a connecting leg 13a³ that is attached to the first leg 13a¹ can be locked into the teeth of the second leg 13a² slides over the second leg 13a². The locking can be disengaged by slightly spreading the two legs 13a¹ and 13a², so that the legs can be more widely opened or closed. By widening the two legs 13a¹ and 13a² completely, the connecting leg 13a³ can be completely decoupled from the teeth and can again be brought into the position shown in FIG. 7c. The principle is known from furniture armatures, e.g. in the form of means for engaging or latching an object in an upward position. The above-mentioned armature can be further modified, e.g. in that the connecting leg 13a³ can also be decoupled manually via the mandrel 13a⁵. In this case, the mandrel 13a⁵ is preferably fixed via an adjusting element such as a thumb nut.

FIG. 8a and 8b show an embodiment of the invention with a movable angle adjusting means 13b. The latter can be made of an elastic or relatively stiff material, depending on the demands. The angle adjusting means 13b can be detachably fixed to at least one of the inner portions 1a, 2a. This is preferably done by means of hook and loop connectors, wherein more preferably both inner portions 1a, 2a are covered with a hook and loop material. Particularly preferably, the angle adjusting means 13b is bent as shown, so that a controlled elastic dampening between the distal element and the proximal element can be achieved. Moreover, in this case a hook and loop connector has to be provided on one side of the bent angle adjusting means 13b. FIG. 8a also shows an initial angle position of 30° between the distal element and the proximal element.

FIGS. 8c to 8e and 8h and 8i show embodiments with link adjusting means 13c and 13d, respectively. In FIGS. 8c to 8e, the depicted link adjusting means 13c is arranged below the distal element and the proximal element. The bottom view according to FIG. 8e shows that the adjustment can easily be performed, e.g., via a corresponding screw 13c′, and that the scaling can be easily realized. FIGS. 8h to 8i show a laterally arranged link adjusting means 13d. By means of the latter, it is also possible to easily adjust the angle but also the distance between the distal element 1 and the proximal element 2.

FIGS. 8f to 8g show a lever/latch mechanism 13f as a further alterative to the angle adjusting means 13.

FIGS. 9a and 9b show a further embodiment of a device of the invention comprising a particularly preferred spreading means 13e. In said embodiment, the distal element 1 and the proximal element 2 are connected via a hinge element 13e¹ preferably having a double hinge. On or in the hinge element 13e¹, there is furthermore a thrust bearing for a threaded spindle 13e⁵ in which the threaded spindle 13e⁵ can freely rotate. A star-shaped grip 13e⁶ via which a user can rotate the threaded spindle is preferably provided on the lower end of the threaded spindle 13e⁵. For the user having enough room for rotating the star-shaped grip 13e⁶ in the folded state (cf. FIG. 9a), the distal element 1 and the proximal element 2 preferably comprise corresponding cavities 1d, 2d.

Moreover, a nut 13e³ is provided, which is held by means of at least One arm 13e⁴ in a non-rotatable state on the threaded spindle 13e⁵ relative to the distal element 1 and proximal element 2. Each arm 13e⁴ is pivotably joined to the corresponding inner portion of the distal element 1 and/or proximal element 2 and the nut 13e³.

Particularly preferably, the threaded spindle 13e⁵ and the nut 13e³ have corresponding fine-pitch threads, more preferably trapezoidal threads, in order to provide a finely adjustable, sufficiently stable arrangement that cannot adjust itself, i.e. is self-locking. By rotating the threaded spindle 13e⁵, the nut 13e³ is moved forwards or backwards on the threaded spindle 13e⁵. The arms 13e⁴ are thus unfolded or folded, and the angle between the distal element 1 and the proximal element 2 is adjusted in a simple but nevertheless effective and permanent manner. FIG. 9a shows a folded state. When rotating the threaded spindle 13e⁵, the nut 13e³ is moved downwards, the arms 13e⁴ are unfolded and thus the distal element 1 is pivoted away relative to the proximal element 2, cf. FIG. 9b.

This arrangement has a plurality of advantages: First, the angle can quickly and Continuously be adjusted, and angles up to 90° can easily be achieved. No tools are necessary therefor. Moreover, the point of rotation for the pivoting movement between the distal element 1 and the proximal element 2 is located at their upper ends and thus as close as possible to and in an ergonomically favorable manner at the shoulder joint of a patient.

Individual features of the above-described different embodiments can be combined with each other.

In sum, the advantages of the invention reside in that the variable arm abduction orthosis provides for an individually adjustable arm length as well as a freely selectable abduction angle. Thus, the orthosis can be put on quickly and easily after an operation already in the recovery room. Moreover, the abduction orthosis offers a great wearing comfort during night and day and can be put on and off by the patient himself/herself without the aid of a third person. It immobilizes the shoulder joint in a physiologically suitable position which can always be adapted to the present rehabilitation state by the therapist and the patient.

1. A device for supporting the arm and/or shoulder comprising a distal element and/or a promixal element as well as an axillary wedge which can be adjusted at least in height relative to the distal element and/or the proximal element.

2. A device for supporting the arm and/or shoulder comprising a distal element as well as a proximal element attached thereto, wherein the distal element and/or the proximal element is/are padded at least in outer portions and essentially rigid at least in inner portions, and wherein an angle adjusting means for adjusting the angle between the distal element and the proximal element is arranged in the area of the inner portions.

3. A device for supporting the arm and/or shoulder according to claim 2 and further comprising an axillary wedge which can be adjusted at least in height relative to at least one of the distal element and the proximal element.

4. A device according to claim 1, wherein the axillary wedge is adjustable in angle in a range of ±15° relative to the distal element and/or the proximal element.

5. A device according to claim 2, wherein the proximal element and the distal element are adjustable relative to each other in distance and angle.

6. A device according to claim 2, wherein the proximal element and the distal element are adjustable relative to each other in distance and angle.

7. A device according to claim 1, wherein at least one adjusting element is provided for adjusting the height, said element being a mechanical element, preferably a telescopic rod, and lockable.

8. A device according to claim 1, wherein the angle adjustment, height adjustment, patient's arm length and/or abduction angle can be directly and/or indirectly read off and/or adjusted.

9. A device according to claim 1, wherein the axillary wedge is adjustable in angle and height, replaceable and/or variable in its outer shape.

10. The device according to claim 2, wherein the connection between distal element and proximal element is realized by a joint-like connection in the upper area of the two elements.

11. The device according to claim 10, wherein the connection between distal element and proximal element is realized by at least one strap, one belt, one joint, one hinge, one strap hinge, one web of fabric or one fixed intermediate roll.

12. The device according to claim 2, wherein the distal element and the proximal element are adjustable relative to each other in an outer angle of at least about 30° to 60°.

13. The device according to claim 2, wherein the distal element and the proximal element are adjustable relative to each other in an outer angle of up to about 90°.

14. The device according to claim 2, wherein the distal element and the proximal element are adjustable relative to each other in an inner angle starting from about 0°.

15. The device according to claim 1, wherein the angle adjusting means is a spreading means comprising a threaded spindle being rotatable by a user, a nut being axially movable on the threaded spindle by rotating the threaded spindle by rotating the threaded spindle, wherein the threaded spindle and/or the nut is/are self-locking, and arms attached to the nut as well as to the distal element and/or proximal element for spreading the distal element and the proximal element.

16. The device according to claim 15, wherein a grip for the handling of the free end of the threaded spindle by the user is provided.

17. The device according to claim 16, wherein the grip is a star-shaped grip.

18. The device according to claim 15, wherein a support for supporting the threaded spindle is attached to each one of the hinge elements arranged on each of the upper ends of the distal element and the proximal element.

19. The device according to claim 18, wherein the hinge element comprises a double hinge, one for the distal element and one for the proximal element.

20. The device according to claim 15, wherein the distal element and the promixal element comprise cavities in order to make room for operating the threaded spindle in the folded state.

21. The device according to claim 2, wherein the angle adjusting means is an engaging or latching means.

22. The device according to claim 21, wherein the two main legs of the engaging or latching means are firmly attached to the inner portions of the distal element and/or the proximal element, the angle can be adjusted in that the latch element engages in teeth and then be increased and/or decreased in little steps, and the engaging or latching means can optionally be disengaged in a maximum position by increasing the angle and can then be folded again.

23. The device according to claim 2, wherein the angle adjusting means is an angle element which is movable between the distal element and the proximal element and which can be firmly and detachably arranged on at least one inner portion of the distal element and/or proximal element.

24. The device according to claim 23, wherein the angle element comprises a first hook and loop means and distal element and/or the proximal element comprise(s) a corresponding second hook and loop means for a firm and detachable arrangement.

25. The device according to claim 2, wherein the angle adjusting means is a link adjusting means.

26. The device according to claim 25, wherein the link adjusting means is essentially arranged at the bottom of the distal element and the proximal element.

27. The device according to claim 25, wherein the link adjusting means is arranged laterally at the distal element and the proximal element.

28. The device according to claim 1, wherein the distal element and/or the proximal element comprise(s) at least one ergonomic cavity.

29. The device according, claim 1, wherein the distal element has a height of about 16 cm, a width of about 10 cm and a length of about 40 cm, the proximal element has a height of about 16 cm, a lower rear width of about 8 cm, a lower front width of about 5 cm, an upper width of about 0.5 to 1 cm and a length of about 40 cm, and the axillary wedge has a height of about 8 cm and a width of about 10 cm, and wherein the overall shape of the orthosis is slightly bent, essentially C-shaped.

30. The device according to claim 1, wherein the axillary wedge has a dorsally raised wedge shape.

31. The device according to claim 1, wherein the distal element, the axillary wedge and/or the proximal element is/are essentially flexible.

32. The device according to claim 1, wherein the distal element and/or the axillary wedge and/or the proximal element is/are padded, e.g., with a foam material with memory effect, such as a relax foam, in particular in the area of the n. ulnaris.

33. The device according to claim 1, wherein the distal element, the axillary wedge and/or the proximal element has/have a steam permeable and liquid repelling cover.

34. The device according to claim 1, wherein the distal element, the axillary wedge and/or the proximal element has/have a removable cover being fixable by means of hook and loop closures.

35. The device according to claim 1, wherein the device comprises a device for fixing the arm, which is at least partially padded.

36. The device according to claim 1, wherein the device can be combined with a hand training device.

37. A set comprising a device according to claim 1, wherein the device comprises at least one first preferably breathable and/or steam permeable cover, and further comprises at least one further cover for postoperatively wearing the device, wherein the further cover is at least liquid repelling.

38. Use of a device according to claim 1 for supporting the arm and/or shoulder in case of injuries, in particular in case of injuries of the shoulder joint, the collar bone and/or the shoulder blade.

39. Use of a device according to claim 32 in case of injuries selected from the group of
shoulder luxations,
ruptures of the rotator muscles,
shoulder head fractures,
shoulder head prostheses,
subcapital humerus fractures,
humerus shank fractures,
AC ruptures, and
scapula fractures close to the joint.

40. Method for treating an injury, in particular an injury of the shoulder joint, collar bone and/or shoulder blade by immobilizing the arm with the aid of a device according to claim 1.
